(19) United States
(12) Patent Application Publication
(10) Pub. No.: US 2023/0054579 A1
(43) Pub. Date: Feb. 23, 2023

(54) PSYCHOMOTOR ABILITY ASSESSMENT METHOD BASED ON MULTI-TARGET TRACKING PARADIGM

(71) Applicant: Civil Aviation University of China, Tianjin (CN)

(72) Inventors: Lei WANG, Tianjin (CN); Jianjun WU, Tianjin (CN); Kengrui SHAO, Tianjin (CN); Zhiyang ZHANG, Tianjin (CN); Shuo WANG, Tianjin (CN); Wei TAN, Tianjin (CN); Shan GAO, Tianjin (CN)

(73) Assignee: Civil Aviation University of China, Tianjin (CN)

(21) Appl. No.: 17/865,057

(22) Filed: Jul. 14, 2022

Related U.S. Application Data
(63) Continuation of application No. PCT/CN2022/074886, filed on Jan. 29, 2022.

(30) Foreign Application Priority Data
Aug. 20, 2021 (CN) 202110958324.6

(51) Int. Cl. A61B 5/16; G06F 3/01

(52) U.S. Cl. CPC A61B 5/162; G06F 3/011; G06F 3/016

(57) ABSTRACT

Disclosed is a psychomotor ability assessment method based on multi-target tracking paradigm, which includes the following steps: analyzing research data of psychomotor ability, determining a plurality of measurement dimensions of psychomotor ability, and designing an experimental task paradigm based on the plurality of measurement dimensions, wherein the experimental task paradigm is used for representing a level of the plurality of measurement dimensions; designing evaluation schemes of the measurement dimension based on the experimental task paradigm; evaluating a ability dimension of a person to be detected based on the evaluation schemes, collecting and processing evaluation data, and calculating a weight value of the ability dimension to obtain an evaluation result of the psychomotor ability.

### CROSS-REFERENCE TO RELATED APPLICATIONS

This application is a continuation of International Application No. PCT/CN2022/074886, filed on Jan. 29, 2022, which claims priority to Chinese Patent Application No. 202110958324.6, filed on Aug. 20, 2021, the contents of which are hereby incorporated by reference.

### TECHNICAL FIELD

The application relates to the field of pilot competency assessment, and in particular to a psychomotor ability assessment method based on a multi-target tracking paradigm.

### BACKGROUND

Aviation safety means no accidents related to aircraft operation, such as casualties and aircraft damage. According to the statistics of civil aviation accidents, human factors are considered to be the main cause of current flight accidents, and crew errors account for two-thirds of all aviation accidents. Civil aviation pilots, as the direct operator of the aircraft, make decisions and conduct operations with a direct impact on the operational safety of the aircraft.

On the other hand, with the development and progress of science and technology, automation and intelligence have become the prominent features of modern aircraft performance, thus putting forward higher standards for the professional competency of pilots. The Professionalism Lifecycle Management System (PLM) put forward by the Civil Aviation of China, is focused on professional competency (three dimensions: core competency, psychological competency and style competency) as the core, driven by empirical trainings and work style construction, based on the evidence input of core competence and psychological assessment of occupational adaptability, and characterized in covering all elements and whole cycle of pilot trainings, and aimed at continuously improving the pilot's ability to guard against “potential crisis with high probability and huge impact” and “high risk event with low probability”, covering related elements such as theory, personnel, equipment, procedures and support system.

Therefore, it is one of the important ways to further enhance the pilot's professional competency, avoid and reduce flight accidents and unsafe incidents by effectively evaluating the individual psychological competency of airline pilots, quantifying and digitizing their abilities, and training and managing pilots accordingly.

There are many factors that affect the psychological competence of airline pilots, and psychomotor ability is included. Psychomotor ability is an important part of psychological quality, one of the important psychological factors in flight skills, and a special ability in the pilot's psychological competency system. Psychomotor ability is very important to the pilot's operational behavior and directly affects pilot's flight performance However, at present domestic and foreign scholars' research on pilots' psychomotor ability is mostly based on the basic elements, which may be summarized into two points: first, the psychomotor ability is comprehensively expounded in theory, but mainly focusing on the factor analysis of psychomotor ability and the factor analysis of influencing psychomotor ability, and the conclusion is relatively one-sided, which is deficient in combining with practice; secondly, there is a lack of experimental paradigm to further strengthen the research intensity and depth, so as to conduct quantitative evaluation. In the research of multi-target tracking, early scholars at home and abroad mainly discussed the regularity of attention phenomenon and the processing mechanism of attention from the aspect of static visual information, such as Bottle Neck model, Attenuation Model, and later Object-based, Location-based, Feature-based and Feature integrated. These studies only focus on the static visual information processing, compared with the real life, the difference between the situational environment and the attention objects is too big, and to some extent, the processing validity is too low. A new paradigm of dynamic information experiment was developed in the later period to strengthen the ecological validity of visual attention processing. Pylyshyn and Storm put forward the multi-target tracking paradigm, which makes a deeper exploration on multi-target tracking and paying attention in a continuous dynamic situation.

### SUMMARY

The objective of the present application is to provide a psychomotor ability assessment method based on a multi-target tracking paradigm so as to solve the problems existing in the prior art, carry out the attention of complex visual information, the processing of perception and cognition, and the movement reaction in a dynamic scene, and comprehensively evaluate the psychomotor ability of pilots, which is of great significance to the evaluation of pilots' special trainings. Meanwhile, the method breaks through the conventional mechanized evaluation mode and proposes the integration of computer evaluation and trainings for individual pilots.

To achieve the above objectives, the present application provides a psychomotor ability assessment method based on the multi-target tracking paradigm, which includes the following steps:

analyzing research data of psychomotor ability, determining a plurality of measurement dimensions of psychomotor ability, and designing an experimental task paradigm based on the plurality of measurement dimensions, of which the experimental task paradigm is used for representing a level of the plurality of measurement dimensions;

designing evaluation schemes of the measurement dimension based on the experimental task paradigm; and

evaluating a ability dimension of a person to be detected based on the evaluation schemes, collecting and processing evaluation data, and calculating a weight value of the ability dimension to obtain an evaluation result of the psychomotor ability.

Optionally, the plurality of measurement dimensions include eye-hand coordination ability, two-hand coordination ability, attention distribution ability and spatial orientation ability.

Optionally, the evaluation schemes include evaluation schemes of eye-hand coordination ability, evaluation schemes of two-hand coordination ability, evaluation schemes of attention distribution ability and evaluation schemes of spatial orientation ability.

Optionally, one of the evaluation schemes of eye-hand coordination ability includes: setting a target and different amounts of distractors, a moving speed of the distractors and a moving boundary between the target and the distractors;

setting a plurality of evaluation scenes based on the distractors and the moving speed, the distractors being moving randomly within the movement boundary in each evaluation scene, and controlling the target to avoid the distractors; after the target touches the distractors or the movement boundary, the test task of the evaluation scene being finished, and recording the duration of the test task of the evaluation scene until all the test tasks of the evaluation scenes are finished, and calculating the total duration of the eye-hand coordination competency assessment.

Optionally, one of the evaluation schemes of two-hand coordination ability includes: setting a left-hand target and a right-hand tracking target with different colors;

the left-hand target appears randomly, and the left hand captures the left-hand target with a corresponding color according to a prompt, and records correct times, while a right hand controls a cursor to track the movement of the right-handed tracking target.

Optionally, one of the evaluation schemes of attention distribution ability includes: setting different numbers of target markers, several appearance positions of points detecting stimulus, presentation modes of visual stimulus and presentation modes of auditory stimulus;

displaying a marker at an initial state in a stimulus presentation area, presenting visual stimulus and auditory stimulus on a specified number of the initial state markers, and obtaining the target markers in a marked state; after finishing the visual stimulus and the auditory stimulus, restoring the target markers in the marked state to the initial state, all the markers being moving randomly, selecting the target markers, and recording a tracking accuracy rate, wherein the number of the target markers in the initial state is greater than a maximum of the number of the target markers.

Optionally, one of the evaluation schemes of spatial orientation ability includes: setting targets with different shapes, positional relationships, moving directions, task target lines and experiment times;

the targets with different shapes appear at the same time, and are arranged according to the positional relationships, and move in the same direction according to different speeds according to the moving directions, and disappear at the same time before reaching the respective task target lines, so as to judge which one of the targets reaches the respective task target line first, and record a reaction time and a accuracy rate until all the experiments are completed, wherein the respective task target lines are on a same straight line.

Optionally, collecting and processing evaluation data includes:

collecting evaluation data, screening the evaluation data, removing abnormal values, deleting data with large deviation by a quartile method, and supplementing default values to obtain effective evaluation data; and

performing a dimensionless standardization processing and a forward processing of reverse indexes on the effective evaluation data.

Optionally, a method of calculating the weight value Wᵢ of each ability dimension is as follows:

$${W}_{i} = \frac{{V}_{i}}{{\sum }_{i = 1}^{n} {V}_{i}} ;$$

where, Vᵢ is a coefficient of variation of i-th index,

$${V}_{i} = \frac{{δ}_{i}}{{\overline{x}}_{i}} ,$$

δᵢ is a standard deviation of i-th index, and x̅ᵢ is an average of i-th index.

The application discloses the following technical effects:

the psychomotor ability assessment method based on multi-target tracking paradigm provided by the application may carry out the attention of complex visual information, the processing of perception and cognition, and the motion response in a dynamic scene, and comprehensively evaluate the psychomotor ability of pilots, which is of great significance to the special training evaluation, breaks through the traditional mechanized evaluation mode, and proposes the integration of computer evaluation and training for individual pilots.

### BRIEF DESCRIPTION OF THE DRAWINGS

In order to more clearly explain the embodiments of the present application or the technical solutions in the prior art, the following will briefly introduce the drawings that need to be used in the embodiments. Obviously, the drawings in the following description are only some embodiments of the present application. For those of ordinary skill in the art, other drawings may be obtained according to these drawings without any creative labour.

FIG. 1 is a flowchart of a psychomotor ability assessment method based on multi-target tracking paradigm in the embodiment of the present application.

FIG. 2 is an overall structure diagram based on the multi-target tracking paradigm provided in the embodiment of the present application.

FIG. 3 is an experimental diagram of eye-hand coordination based on multi-target tracking paradigm provided in the embodiment of the present application, in which (a) is original state; (b) shows that the testee manipulates the target to avoid distractor for ; (c) shows that the target collides with the distractor-the ccollision is over.

FIG. 4 is an experimental diagram of two-hand coordination based on multi-target tracking paradigm provided in the embodiment of the present application.

FIG. 5 is an experimental diagram of attention allocation based on multi-target tracking paradigm provided in the embodiment of the present application, in which (a) is preparation stage; (b) is fixation point (1s); (c) is stimulus presentation; (d) is clue stage; (e) is tracking stage; (f) is reaction stage.

FIG. 6 is an experimental diagram of spatial orientation based on multi-target tracking paradigm provided in the embodiment of the present application.

FIG. 7 is a data acquisition diagram of the multi-dimensional psychomotor ability assessment method provided in the embodiment of the present application.

### DETAILED DESCRIPTION OF THE EMBODIMENTS

Compared with the prior art, the embodiment of the application has the following innovative effects: the embodiment of the application provides an integrated method for evaluating and training individual pilots' psychomotor ability, which breaks through the traditional mechanized evaluation mode. By designing task test content based on multi-target tracking paradigm, the method may perform attention to complex visual information, process and motion response of perception and cognition in a dynamic scene; long-term continuous attention through multi-target tracking, combined with the pilot's task characteristics to ensure effective tracking of the object target and effective attention processing of the object target information; through the dynamic visual processing of tracking and observing multiple object targets at the same level, the ecological validity of comprehensive evaluation of pilots' psychomotor ability may be improved, and the test results have important guiding significance for special training.

The technical solutions in the embodiments of the present application will be clearly and completely described below with reference to the drawings in the embodiments of the present application. Obviously, the described embodiments are only part of the embodiments of the present application, but not all of them. Based on the embodiment of the present application, all other embodiments obtained by ordinary technicians in the field without creative labor are within the scope of the present application.

In order to make the above objectives, features and advantages of the present application more obvious and understandable, the present application will be explained in further detail below with reference to the drawings and detailed description.

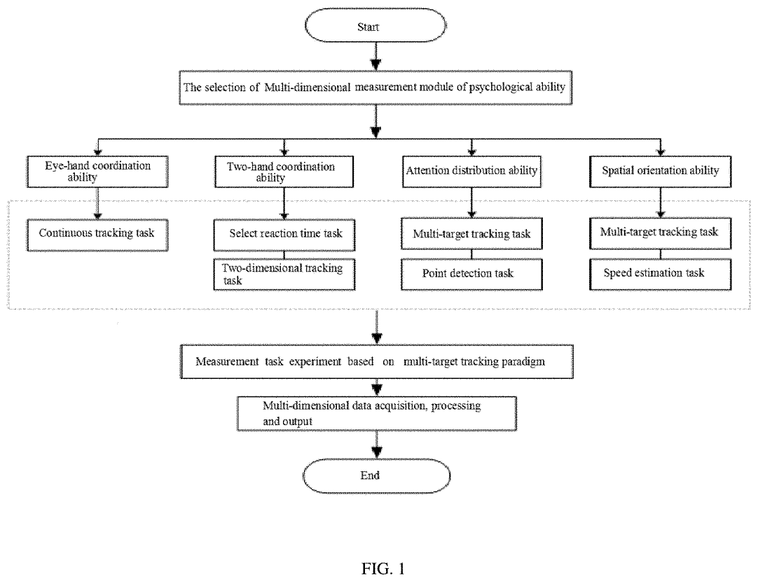

The application provides a psychomotor ability assessment method of multi-target tracking paradigm, which is shown in FIG. 1 and includes the following steps:

Step 1, determining the measurement dimension of pilot's psychomotor ability through the analysis of domestic and foreign literatures.

By summarizing the research on pilots' comprehensive ability, psychological competence, cognitive ability, psychological evaluation and other related indexes by domestic and foreign scholars and exploring the correlation between basic elements of psychomotor ability and flight operation behavior, determining the definition of factors that may affect pilots' psychomotor ability.

Determining the four dimensions indexes: the four dimensions of eye-hand coordination ability, two-hand coordination ability, attention distribution ability and spatial orientation ability through the above literature research results.

Step 2: selecting the multi-target tracking experimental paradigm to design the psychomotor ability measurement scheme according to the research and analysis of domestic and foreign literatures and the determined dimensions of pilots' psychomotor ability measurement, and selecting different performance indexes for output according to the test results of the psychomotor ability measurement scheme.

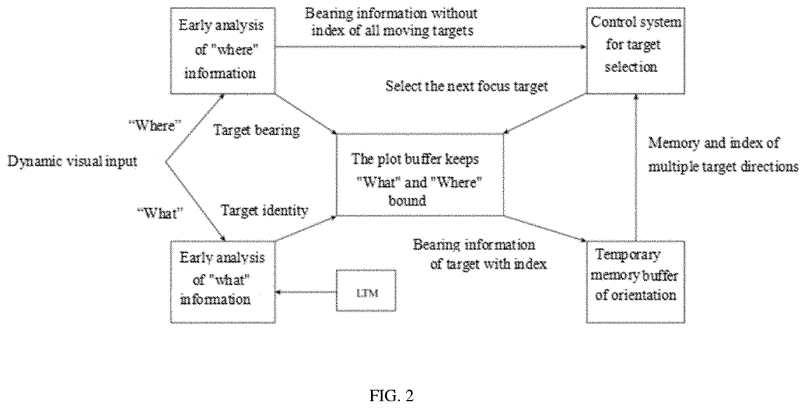

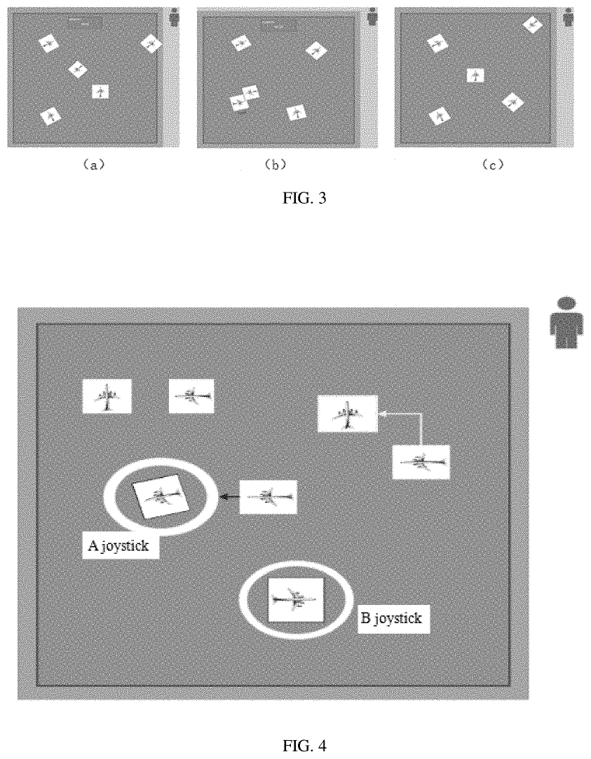

In this embodiment, according to the characteristics of the multi-target tracking experimental paradigm, as shown in FIG. 2: the multi-target tracking task requires long-term continuous attention, rather than simple attention transfer; multi-target tracking task involves paying attention to multiple targets, rather than focusing on a single target at a time; multi-target tracking task is a data-driven task, which is activated spontaneously and needs no preparation; in multi-target tracking task, attention capacity may be directly quantified by the change of tracking quantity, which is a direct way; the above characteristics completely accords with the characteristics of visual cognition in the real world. For example, in real situations, all kinds of driving activities need to pay continuous attention to the movement changes of multiple targets over time. On the basis of the Step 1, combined with the characteristics of the dynamic situation of the flight mission, a multi-target tracking experimental paradigm is selected to explore the visual attention and memory processing process of multiple targets in the dynamic situation. This experimental paradigm usually includes three stages: clue stage, tracking stage and reaction stage. Taking the multi-target tracking experimental paradigm as the core, based on the input of four measurement dimensions: eye-hand coordination, two-hand coordination, attention distribution and spatial orientation, a measurement scheme matching with each capability dimension is designed. The concept demonstration is shown in FIG. 3-FIG. 6.

The measurement scheme of eye coordination ability is shown in FIG. 3:

The measurement is preset to two factors: 3 (moving speed: high-speed 7.5°/s, medium-speed 6.5°/s, low-speed 5.5°/s)×3 (number of distractors: 4, 5, 6) in-group design, which is refreshed once in 16 ms (one frame). The tracking duration of all experimental attempts of the testee is recorded and the weighted average (ms) is calculated as the final task performance index.

The specific calculation method is shown as Formula (1):

$$\begin{matrix}T = \frac{{\sum }_{i = 1}^{n} {t}_{i} {f}_{i}}{n} , & ( i = 1 , 2 , 3 , … n )\end{matrix} \qquad ( 1 )$$

where T is the final task performance value, tᵢ is the tracking duration of the i-th attempt, ƒᵢ is the weight, Σᵢ₌₁ⁿ is the frequency of tᵢ appearance, and n is the total number of experimental attempts.

Rest for 3 minutes before the measurement starts. There are 10 times Under each experimental condition. The testee first completes the high-speed, medium-speed and low-speed tasks with four distractors, then completes the tasks with three speeds with five distractors, and finally completes the tasks with three speeds with six distractors. After completing the task of each distractor condition, the testee may rest for 2 minutes. The target is the red airplane model, and the distractor is the green airplane model. All objects are presented in a 360×360 pixel gray frame with a sky blue background. The dark gray border in the gray border is the boundary where the red plane can't touch, and the distractor may move here. The testee controls the movement of the target with a joystick. After clicking on the target, the distractors begin to move, and the trajectories of the distractors are linear and independent of each other. The moving speed of the distractor is not constant, and the position of the object in each frame is determined by the position and moving speed of the previous frame. The moving speed of the distractor randomly changes with the probability of 5% in each frame, and the changing range is the initial speed±2. During the movement, the distractors may pass through each other but will bounce when they touch the gray border. When the target touches the distractor or the dark gray border, the task ends, and the time for the testee to complete the task is displayed in the center of the plane. The time for the testee to complete the whole task depends on the continuous tracking time.

Measurement scheme of two-hand coordination ability, as shown in FIG. 4:

the experiment is preset with two factors: 3 (moving speed: high-speed, medium-speed, low-speed)×3 (number of distractors: 4, 5, 6) in-group design, and it is refreshed once in 16 ms (one frame). The accuracy rate of the testee and the reaction time of all attempts under each experimental condition are recorded, and the weighted average value of the reaction time (ms) is taken as the final task performance index.

The specific calculation method is shown in Formula (2):

$$\begin{matrix}M = \frac{{\sum }_{i = 1}^{n} {m}_{i} {j}_{i}}{n} , & ( i = 1 , 2 , 3 , … n )\end{matrix} \qquad ( 2 )$$

where M is the performance value of the final task, mᵢ is the i-th reaction time, jᵢ is the weight, Σᵢ₌₁ⁿ is the frequency of mᵢ, and n is the total number of experimental attempts. The measurement is divided into two tasks, the action selection reaction time measurement and the two-dimensional tracking test, which are completed by the left and right hands respectively, and the two tasks are carried out simultaneously. Ten attempts are conducted under each experimental condition.

The testee operates the bar A with left hand to complete the action selection reaction time test, and operates the bar B with his right hand to complete the two-dimensional tracking test. If three kinds of color target aircraft (red, yellow and green) randomly appear on the screen, the testee is required to judge whether it is the color target (red and yellow) that needs to be confirmed by operating the A-bar (i.e., the control ring of the A-bar is manipulated to select the red target, and the system responds after clicking the confirm key); if there is a green target, there is no need to manipulate the A-bar for action; then the right hand manipulates the circular cursor through the B-bar to coincide with the blue airplane model (free movement, irregular speed change) as much as possible. If not coincide, the system will sound an alarm (need to be corrected immediately), and follow the rectangular area to make a plane movement. The left and right hands operate at the same time to complete their respective tasks without interfering with each other.

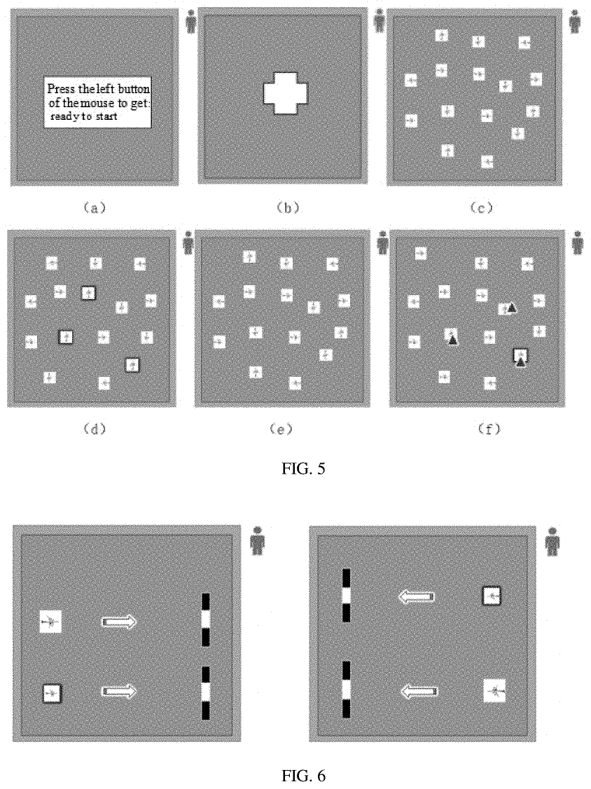

Measurement scheme of attention distribution ability, as shown in FIG. 5:

The dual-task experimental paradigm of multi-target tracking task and point detection task is adopted. The experiment is preset with two factors: 5 (number of targets: 3, 4, 5, 6, 7)×3 (appearance positions of points detecting stimulus: target position, empty position and non-target position) in-group design, and it is refreshed once in 16 ms (one frame). The tracking accuracy rate and point detection perceiving rate of the testee are recorded as the the final task performance index. Where the tracking accuracy rate refers to: calculating the percentage of correctly selecting target in all experimental attempts of the testee under the condition of different target numbers; perceiving rate refers to the ratio of the number of times that the testee correctly perceived the detecting stimulus to the total number of times that the detecting stimulus appear.

Multi-target tracking task adopts (visual+auditory) double stimulus mode, visual stimulus and auditory stimulus are presented at the same time. The subjects screen the visual information and auditory information respectively according to the screening requirements. Visual information presents “target action information (flashing)”, auditory information presents “target color information (red)”, and combined with the double information, the target “flashing red airplane model” is searched. Point detection task: during the main task experiment, the detecting stimulus red dots randomly appear at the target, non-target and blank area, and the presentation time is preset to 200 ms. If the testee finds a red dot, after finishing the main task, a question will appear on the screen whether a red dot is found. If so, press “Y” and if not, press “N”. After the testee makes a choice, press the left button of the mouse to enter the next attempt. In the process of object movement, each object will be sheltered from each other without using collision detection algorithm, but the object presenting point detecting stimulus is always at the front end (i.e., the point detecting stimulus will not be blocked). In the experiment, the red spots appeared 30 times, and the areas where the red spots appeared were evenly distributed in the target position, the non-target position and the blank position. Each area will appear 10 times. The index is recorded as the point detection perceiving rate at each position.

Before the test, conduct five pretesting tests to familiarize the testee with the test process. The testee is about 60 cm away from the screen, and the stimulus presentation area is the whole screen. When the experiment starts, “+”appears in the middle of the screen for 1000 ms, then 15 white airplane models are displayed on the screen; 3, 4, 5, 6 and 7 airplane models under different attempt conditions changed from white to red and move in the direction specified by the system, and flash for 3 times, marked as targets, and the rest of them with no color changea ere non-targets. After that, all airplane models returned to white. Then, the model starts to move in random directions (up, down, left and right) at the angle of view of 5°/s. During the movement, shelter occurs from time to time, and the system prompts to search for the target information. After 20 s, the model stops moving, and the testee is asked to point out the target with the left button of the mouse, and then the next attempt. According to the number of preset color-changing airplane models in the test, the test is divided into “blocks”. The test consists of 5 “blocks”, each of which has 10 tracking times, with a total of 50 attempts. Each of the “blocks” is separated by 3 min, and the whole test lasts for about 30 min. The index is recorded as tracking accuracy rate.

Measurement scheme of spatial orientation ability, as shown in FIG. 6:

The experimental paradigm of multi-target tracking task and relative arrival time judgment task is adopted. The experiment is preset with three factors: 2 (target characteristics: size and speed are not set repeatedly)×2 (target position relationships: up-down relationship, left-right relationship)×4 (target moving direction: from left to right, from right to left, from top to bottom, from bottom to top). The response time and accuracy rate of the testee are recorded as the final performance indexes.

The basic stimulus of this experiment: two airplane models of different sizes appear on the display screen at the same time, one up and one down (one left and one right) move horizontally and linearly at different speeds but in the same direction, and each of them moves towards the opposite target line, then disappears (does not reach the target line) after moving for a period of time. It is basically set as a colorful airplane model on the sky blue background, with one big model and one small model, one red and one green. The two target lines are black and white, and the vertical line from the airplane model to the target line is the midpoint of the target line. The two target lines have the same position in the display (i.e., they may be connected into a straight line). Speed: the model moving speed may be divided into two levels (high-speed and low-speed). On the positional relationship: one left and one right, one up and one down. In the movement direction: from left to right, from right to left (up-down relation), from top to bottom, from bottom to top (left-right relation). Ten attempts are conducted under each experimental condition. In the experimental task, the testee needs to judge which one of the red and green models reaches the target line first (set to: the model will continue to move at the original speed after it disappears.), and operate the left and right buttons on the joystick to confirm (left red and right green) respectively. The dependent variables are reaction time and accuracy, and the timing starts when the model disappears, and ends when the button reacts.

According to the test results, the following performance indexes are obtained: the continuous tracking time of eye-hand coordination ability, the response time and accuracy of two-hand coordination ability, the accuracy rate and perceiving rate of attention distribution ability, and the response time and accuracy rate of spatial orientation ability.

3. Using the test scheme designed in Step 2, perform measurements of eye-hand coordination ability, two-hand coordination ability, attention distribution ability and spatial orientation ability on the pilot, and obtain measurement data, and preprocess the obtained test data.

The specific steps are as follows:

Screen the measured data, eliminate the abnormal values, delete the data with large deviation by quartile method, and supplement it according to the default value. In this embodiment, the process of determining the data with large deviation by using the quartile method is as follows: the lower quartile of the data refers to the value (Q1) corresponding to 25% of the data; median is the value (Q2) corresponding to the 50% fractile; the upper quartile is the value corresponding to 75% fractile of the data (Q3); the calculation formula of upper must value is Q3+1.5 (Q3−Q1); the calculation formula of lower must value is Q1−1.5 (Q3−Q1). In which Q3−Q1 represents quartile deviation. The criterion is that when the data value of a variable is greater than the upper must value or less than the lower must value, it is considered that such data points have a large deviation.

The acquired multi-dimensional ability test data is processed by Z-score in dimensionless standardization, followed by the forward processing of reverse indexes;

Positive index standardization is shown in Formula (3):

Y=(X−Xₘᵢₙ)/(Xₘₐₓ−Xₘᵢₙ) (3)

where, Y is the normalized value of positive indexes (accuracy rate and perceiving rate), X is the normal value of indexes, Xₘₐₓ is the maximum value of indexes, and Xₘᵢₙ is the minimum value of indexes.

Reverse index standardization is shown in Formula (4):

Z =(Pₘₐₓ−P)/(pₘₐₓ−Pₘᵢₙ) (4)

where, Z is the normalized value of reverse indexes (tracking time and reaction time), P is the normal value of indexes, Pₘₐₓ is the maximum value of indexes, and Pₘᵢₙ is the minimum value of indexes.

Positive index: the larger the value, the better the performance; reverse index: the smaller the value, the better the performance.

Calculate the weight of each dimension ability according to the coefficient of variation method, and finally get the total score of psychomotor ability measurement.

The variation coefficient of each measurement item is shown in Formula (5):

$$\begin{matrix}{V}_{i} = \frac{{δ}_{i}}{{\overline{x}}_{i}} , & ( i = 1 , 2 , 3 , … n )\end{matrix} \qquad ( 5 )$$

where, Vᵢ is the coefficient of variation of i-th index, also known as the coefficient of standard deviation; δᵢ is the standard deviation of i-th index; x̅ᵢ is the average of the i-th index.

The weight Wᵢ of each index is calculated as shown in Formula (6):

$$\begin{matrix}{W}_{i} = \frac{{V}_{i}}{{\sum }_{i = 1}^{n} {V}_{i}} , & ( i = 1 , 2 , 3 , … n )\end{matrix} \qquad ( 6 )$$

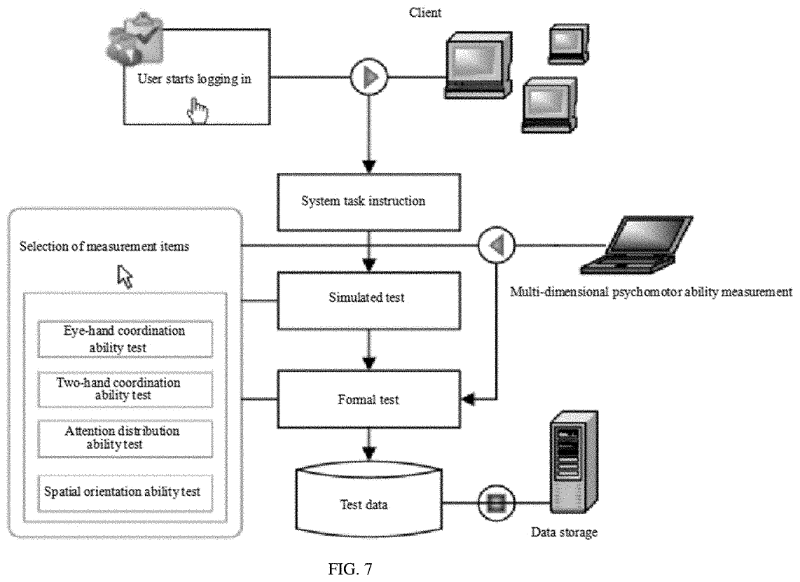

FIG. 7 shows the acquisition of multi-dimensional capability measurement data, and the capability of each measurement dimension is obtained by operating the experimental task based on multi-target tracking paradigm to obtain the corresponding performance indexes.

The psychomotor ability assessment method based on multi-target tracking paradigm is to construct the evaluation system of pilots' psychomotor ability by combining the characteristics of multi-target tracking experiment paradigm and dynamic situation of flight mission. Compared with the traditional mechanized single evaluation, the evaluation system of pilots' psychomotor ability is constructed in many dimensions and aspects, which is more convincing in training and evaluation of pilots' psychomotor ability.

The above-mentioned embodiments only describe the preferred mode of the application, but do not limit the scope of the application. On the premise of not departing from the design spirit of the application, all kinds of modifications and improvements made by ordinary technicians in the field to the technical scheme of the application shall fall within the scope of protection determined by the claims of the application.

What is claimed is:

1. A psychomotor ability assessment method of a multi-target tracking paradigm, comprising:
analyzing research data of a psychomotor ability, determining a plurality of measurement dimensions of the psychomotor ability, and designing an experimental task paradigm based on the plurality of measurement dimensions, wherein the experimental task paradigm is used for representing a level of the plurality of measurement dimensions;
designing evaluation schemes of the plurality of measurement dimensions based on the experimental task paradigm; and
evaluating ability dimensions of a person to be tested based on the evaluation schemes, collecting and processing evaluation data, and calculating a weight value of the ability dimensions to obtain an evaluation result of the psychomotor ability.

2. The psychomotor ability assessment method of the multi-target tracking paradigm according to claim 1, wherein the plurality of measurement dimensions comprise an eye-hand coordination ability, a two-hand coordination ability, an attention distribution ability and a spatial orientation ability.

3. The psychomotor ability assessment method of the multi-target tracking paradigm according to claim 2, wherein the evaluation schemes comprise evaluation schemes of the eye-hand coordination ability, evaluation schemes of the two-hand coordination ability, evaluation schemes of the attention distribution ability and evaluation schemes of the spatial orientation ability.

4. The psychomotor ability assessment method of the multi-target tracking paradigm according to claim 3, wherein one of the evaluation schemes of the eye-hand coordination ability comprises: setting a target and different amounts of distractors, a moving speed of the distractors and a moving boundary between the target and the distractors;
setting a plurality of evaluation scenes based on the distractors and the moving speed, the distractors being moving randomly within the movement boundary in each evaluation scene, and controlling the target to avoid the distractors; after the target touches the distractors or the movement boundary, the test task of the evaluation scene being finished, and recording the duration of the test task of the evaluation scene until all the test tasks of the evaluation scenes are finished, and calculating the total duration of the eye-hand coordination competency assessment.

5. The psychomotor ability assessment method of the multi-target tracking paradigm according to claim 3, wherein one of the evaluation schemes of the two-hand coordination ability comprises: setting a left-hand target and a right-hand tracking target with different colors;
wherein the left-hand target appears randomly, and the left hand captures the left-hand target with a corresponding color according to a prompt, and records correct times, while a right hand controls a cursor to track the movement of the right-handed tracking target.

6. The psychomotor ability assessment method of the multi-target tracking paradigm according to claim 3, wherein one of the evaluation schemes of attention distribution ability comprises: setting different numbers of target markers, several appearance positions of points detecting stimulus, presentation modes of visual stimulus and presentation modes of auditory stimulus;
displaying a marker at an initial state in a stimulus presentation area, presenting visual stimulus and auditory stimulus on a specified number of the initial state markers, and obtaining the target markers in a marked state; after finishing the visual stimulus and the auditory stimulus, restoring the target markers in the marked state to the initial state, all the markers being moving randomly, selecting the target markers, and recording a tracking accuracy rate, wherein the number of the target markers in the initial state is greater than a maximum of the number of the target markers.

7. The psychomotor ability assessment method of the multi-target tracking paradigm according to claim 3, wherein one of valuation schemes of the spatial orientation ability comprises: setting targets with different shapes, positional relationships, moving directions, task target lines and experiment times;
the targets with different shapes appear at a same time, and are arranged according to the positional relationships, and move in the same direction according to different speeds according to the moving directions, and disappear at the same time before reaching the respective task target lines, so as to judge which one of the targets reaches the respective task target line first, and record a reaction time and a accuracy rate until all the experiments are completed, wherein the respective task target lines are on a same straight line.

8. The psychomotor ability assessment method of the multi-target tracking paradigm according to claim 1, wherein collecting and processing evaluation data comprises:
collecting evaluation data, screening the evaluation data, removing abnormal values, deleting the data with a large deviation by a quartile method, and supplementing default values to obtain effective evaluation data; and
performing a dimensionless standardization processing and a forward processing of reverse indexes on the effective evaluation data.

9. The psychomotor ability assessment method of the multi-target tracking paradigm according to claim 1, wherein a method for calculating a weight value Wᵢ of each ability dimension is as follows:
$${W}_{i} = \frac{{V}_{i}}{{\sum }_{i = 1}^{n} {V}_{i}}$$
wherein Vᵢ is a coefficient of variation of i-th index,
$${V}_{i} = \frac{{δ}_{i}}{{\overline{x}}_{i}} ,$$
δᵢ is a standard deviation of i-th index, and x̅ᵢ is an average of i-th index.
